(19) United States
(12) Patent Application Publication
(10) Pub. No.: US 2010/0043143 A1
(43) Pub. Date: Feb. 25, 2010

(54) LOW AIR LOSS MOISTURE CONTROL MATTRESS OVERLAY

(75) Inventors: James R. O'Reagan, Greer, SC (US); Joseph A. Lazich, Jr., Greenville, SC (US)

Correspondence Address: DORITY & MANNING, P.A., POST OFFICE BOX 1449, GREENVILLE, SC, 29602-1449, US

(73) Assignee: SPAN-AMERICA MEDICAL SYSTEMS, INC., Greenville, SC (US)

(21) Appl. No.: 12/607,215

(22) Filed: Oct. 28, 2009

Related U.S. Application Data
(62) Division of application No. 12/110,702, filed on Apr. 28, 2008.
(60) Provisional application No. 60/926,875, filed on Apr. 30, 2007.
(60) Provisional application No. 61/019,723, filed on Jan. 8, 2008.

(51) Int. Cl. A47C 21/04; A61F 7/00

(52) U.S. Cl. 5/421

(57) ABSTRACT

Disclosed are apparatus and methodology for controlling humidity (i.e., moisture) within and/or adjacent a coverlet associated with a multi-layer air mattress. Humidity sensors may be incorporated at selected locations within the coverlet and coupled to a controller that is configured to provide a closed-loop control signal to vary air flow rate through the coverlet to control the rate of moisture adjustment, such as removal. In part, present subject matter may include a combination of a Low-Air-Loss (LAL) topper, having a pair of humidity sensors, one of which is at an inlet to the LAL topper and the other of which is at a selected one of existing outlets of the LAL topper. Air flow through the LAL topper is kept constant, in accordance with its basic operation, while the two humidity sensors are part of a closed-loop feedback control system, which adjusts the humidity of the air being input at the inlet. Additionally portions of a closed-loop feedback control system may depend on the sensed rate of change of moisture, to facilitate more rapid response to changing moisture conditions. Separately, and not as part of the control feedback loop, a temperature sensor may be provided for simply indicating the temperature of the air relative to the LAL topper.

### PRIORITY CLAIM

This application is a divisional of U.S. patent application Ser. No. 12/110,702 filed Apr. 28, 2008 entitled “LOW AIR LOSS MOISTURE CONTROL MATTRESS OVERLAY” which, in turn, claimed benefit of U.S. Provisional Applications bearing the same title and respectively assigned U.S. Ser. No. 60/926,875, filed Apr. 30, 2007, and U.S. Ser. No. 61/019,723, filed Jan. 8, 2008, all of which are incorporated herein by reference for all purposes.

### FIELD OF THE INVENTION

This subject matter generally relates to mattresses and mattress coverlets for preventing, reducing, and/or treating decubitus ulcers, also known as pressure sores or bedsores. More particularly, this subject matter concerns mattresses or mattress coverlets capable of monitoring and controlling moisture levels related to a patient.

### BACKGROUND OF THE INVENTION

Often, patients that are bedridden or immobile can develop decubitus ulcers (pressure sores or bedsores). Such ulcers are often caused by pressure, friction, shear forces, moisture, and/or heat. Pressure results in a reduction of blood flow to the soft tissues of the body, particularly the skin. Continuous lack of blood flow, and the resultant lack of oxygen, can cause the skin to die or atrophy, and cause ulcers or sores to form. Friction and shear of the skin against the support surface can lead to skin tears and decubitus ulcers. Moisture and heat may lead to skin maceration. Other factors play a part in determining the speed with which such ulcers will either tend to form or heal, including such as the overall health of the patient and such patient's nutritional status.

To insure normal (or, at least, relatively improved) blood flow to such areas of potentially problematic contact, patients are often regularly turned or repositioned by medical personnel. Turning or repositioning of patients, however, is not always possible, particularly where trained medical staff is not available. Additionally, repositioning can be painful and disruptive for the patient.

In an effort to overcome such difficulties, a number of mattresses and mattress coverlets have been developed with the intention of more evenly distributing, across the patient's skin, the pressure generated by the weight of the body. At least two methods have been used to redistribute skin pressure. The first is the use of static supports such as foam, air or water mattresses. The second method involves the use of alternating pressure inflatable mattresses or mattress coverlets that dynamically shift the location of support under the patient. Two examples of alternating pressure inflatable surfaces are illustrated in U.S. Pat. Nos. 5,509,155 and 5,926,884.

In addition to such two methods of redistribution of skin pressure, an additional feature has been utilized to help address other of the aforementioned factors important to the healing process. In particular, a low air loss feature has been used to aid in the removal of both moisture vapor and heat, thereby reducing both at the patient-bed boundary. This has been done in an effort to prevent skin maceration, keep wounds dry, and promote healing.

There have been at least three approaches to achieving a low air loss support surface. First, relatively tiny holes can be provided in the top surface of inflatable air cells of an air mattress having a vapor-permeable top surface. Such holes allow extra air to circulate inside the mattress to assist in drying moisture vapor otherwise passing through the top surface from the patient.

Second, relatively tiny holes can be provided in the top surface of the mattress so that the air venting from the air cells can transfer through the top surface to the patient in order to remove both heat and moisture from the area immediately surrounding the patient.

Finally, a multi-layer mattress coverlet can be used wherein the top layer is perforated to allow air flowing between the top layer and a middle vapor-permeable layer to exhaust across the patient, thus aiding in removing both moisture and heat from the area immediately surrounding the patient. The third layer of such a three-layer approach may be a three-dimensional fabric, which allows for additional moisture vapor to be carried away from the patient.

While each of these approaches is useful for its purpose, there are various disadvantages with these approaches and in particular, with using them individually. The first and second referenced approaches to obtaining a low air loss feature require a relatively large compressor pump or the like to maintain sufficient air to inflate the air cells of the mattress. Such large compressor pumps tend to be very noisy, require high electrical consumption, and generate significant heat in a relatively confined area. Such high electrical consumption, and the additional need for continuous blower operation, has, in the past, resulted in potential over-heating of the air used to circulate about the patient. Conversely, in the case of an elderly patient, airflow directly across their body could result in an uncomfortable reduction in body temperature or even a drying out of the skin beyond that which is helpful.

Additionally, having holes in air cells of an inflatable air system results in a support surface that will deflate if there is a loss of electrical power or if no such power supply is available. Further, having perforations in the patient-bed contact surface results in a mattress that is not fluid-proof. Such arrangement allows for potential contamination of the interior of such mattress by bodily fluids, products used to treat the patient, and/or products used to clean such mattress itself. All three referenced approaches generally fail in some respects to allow air to flow under the load (i.e., underneath the patient) or through the top surface to the patient's skin when supporting the weight of the patient.

Similarly, some prior art mattresses and mattress coverlets have had difficulty in controlling billowing. Billowing is generally the uncontrolled inflation of the upper surface of a mattress or mattress coverlet in the area immediately surrounding the outline of a patient's body when the patient lies on the mattress. In essence, the mattress or mattress coverlet fails to fully support a patient and instead seemingly envelops them when the patient's weight is applied thereto. Thus, such billowing further illustrates the failure of some prior mattresses and/or mattress coverlets to fully support the patient, therefore resulting in air flow through the mattress, mattress top layer, or through the coverlet (i.e., the three aforementioned approaches) and around the patient, rather than flowing underneath the patient to aid in controlling moisture and heat.

With all of the above approaches, it is further unknown to have the capability to turn on or off the low air loss option while retaining (through the use of powered air cells) the features of the mattresses or mattress coverlets relating to redistribution of skin pressure. If a low air loss therapy is not desired, a different system must be utilized with an alternative controller and air cell array.

Various aspects of the prior art are described in the following exemplary-only issued U.S. patents. Stolpmann (U.S. Pat. No. 6,855,158) discloses in part a closed-loop control system for support surface temperature control, used in conjunction with a low air loss mattress. Harrison et al. (U.S. Pat. No. 6,859,967) discloses a mattress overlay and various air inflated bladders incorporating thermal control to regulate a patient's body temperature while also using pressure shifting techniques to reduce the risk of bed sore formation.

Gazes (U.S. Pat. No. 5,970,550) discloses a multiple compartment inflatable mattress which involves controlling the temperature of a circulated medium in order to control the mattress temperature. Stroh et al. (U.S. Pat. No. 5,168,589) discloses a pressure reduction air mattress (or alternatively an overlay) which uses adjustable air flow rates as well as heating elements for warming air passed therethrough or thereby. Heaton (U.S. Pat. No. 6,730,115) provides an inflatable mattress and related heat exchanger technology, intended in part for providing cooling contact for a person supported thereon, rather than heating, in order to provide cooling as part of a clinical treatment. Totton et al. (U.S. Pat. No. 6,782,574) relates to an air-powered low interface pressure support surface in which an air inflatable mattress and mattress coverlet are provided for the prevention and treatment of decubitus ulcers (i.e., pressure sores or bedsores). The disclosures of all of the foregoing U.S. patents are fully incorporated herein by reference, for all purposes.

While various implementations of therapeutic mattresses or mattress coverlets have been developed, no design has emerged that generally encompasses all of the desired characteristics as hereafter presented in accordance with the subject technology.

### SUMMARY OF THE INVENTION

In view of the recognized features encountered in the prior art and addressed by the present subject matter, improved apparatus and methodology for monitoring and controlling moisture within a therapeutic mattresses or mattress coverlet have been provided.

In an exemplary configuration, therapeutic mattresses or mattress coverlets are provided with an automatic air flow control system to control the amount of moisture within the therapeutic mattresses or mattress coverlet.

In one of their simpler forms, moisture sensors are appropriately positioned within a therapeutic mattresses or mattress coverlet to monitor the amount of moisture contained in the air circulating through the therapeutic mattresses or mattress coverlet.

Another aspect of the present subject matter (including devices and methodology) is that the level of moisture within the therapeutic mattresses or mattress coverlet (that is, the humidity therein) may be automatically controlled.

In accordance with aspects of certain embodiments of the present subject matter, methodologies are provided to control the relative humidity of air circulating within a therapeutic mattresses or mattress coverlet by reference to either or both of the moisture level and temperature of the circulating air. Still further, other present embodiments may provide feedback control with reference to the rate of change of sensed conditions, such as sensed moisture (or humidity).

In accordance with aspects of certain other embodiments of the present technology, apparatus and methodologies are provided to automatically adjust the rate of air flow within a therapeutic mattresses or mattress coverlet to control the amount of moisture entrained in the flowing air within the therapeutic mattresses or mattress coverlet.

In accordance with yet still further embodiments of the present technology, alternative methodologies for either of adding and/or removing moisture entrained in the air flow within a therapeutic mattresses or mattress coverlet may include, without being limited thereto, active humidification and/or dehumidification.

Disclosed and practiced in various present embodiments are apparatus and methodology for controlling humidity (i.e., moisture) within a coverlet associated with a multi-layer air mattress. More particularly, in exemplary some embodiments, humidity sensors may be incorporated at selected locations within the coverlet and coupled to a controller that is configured to provide a closed-loop control signal to vary air flow rate through the coverlet to control the rate of moisture adjustment, such as removal.

Still further, in part, present subject matter may include a combination of a Low-Air-Loss (LAL) topper, having a pair of humidity sensors, one of which is at an inlet to the LAL topper and the other of which is at a selected one of existing outlets of such an LAL topper. Air flow through such exemplary LAL topper may preferably be kept constant, in accordance with its basic operation, while the two humidity sensors may be provided as part of a closed-loop feedback control system, which preferably adjusts the humidity of the air being input at the inlet. Separately, and not as part of the control feedback loop, a temperature sensor may be provided for simply indicating the temperature of the air relative to the LAL topper.

Yet still further, the present subject matter may include a control circuit coupled to or provided as a part of an existing controller and configured to monitor the rate of change of any detected humidity (moisture) related to a patient.

One exemplary embodiment of the present subject matter relates to an apparatus for regulating humidity of air circulating relative to a patient support, comprising a mattress coverlet, a plurality of humidity sensors, a coverlet air pump, and a controller. Preferably, such mattress coverlet is for association with a main patient support structure, and defines at least one air inlet thereof and at least one air outlet thereof. Such plurality of humidity sensors are preferably coupled respectively adjacent to such mattress coverlet air inlet and such air outlet, so as to detect humidity adjacent such air inlet and air outlet, and so as to provide respective output signals indicative of each. Such coverlet air pump may be coupled to such coverlet and configured to move air inside such coverlet from such air inlet thereof to such air outlet. Such controller preferably is coupled to such coverlet air pump and such respective output signals of such humidity sensors, for controlling the humidity of the air moved inside such coverlet.

In the foregoing exemplary embodiment, such controller, such coverlet air pump, and such humidity sensors may be arranged so as to comprise a closed loop control system. In some embodiments, such controller may be configured for adjusting operation of such coverlet air pump so as to adjust the flow rate of air moved inside such coverlet, while in others it may be configured for adjusting the humidity of air output by such coverlet air pump so as to adjust the humidity of air moved inside such coverlet. In yet others, such controller may be configured for determining relative humidity of air based on such humidity sensor output signals, and adjusting the humidity of air output by such coverlet air pump so as to adjust the relative humidity of air moved inside such coverlet.

In other present exemplary embodiments of the foregoing, such controller may be configured for determining the rate of change of the humidity of air based on such humidity sensor output signals, and adjusting the output of such coverlet air pump so as to adjust the humidity of air moved inside such coverlet, and such exemplary apparatus may further include an indicator associated with such controller, for indicating whenever the rate of change of the humidity exceeds predetermined levels, to indicate possible hemorrhaging or incontinence of a patient.

For some exemplary embodiments, the foregoing exemplary plurality of humidity sensors may include three sensors, at least one of which is adjacent such mattress coverlet air inlet. In some embodiments, there may be at least two such mattress coverlet air outlets, and at least two of such plurality of humidity sensors situated respectively adjacent such at least two mattress coverlet air outlets. In such exemplary instance, such controller is configured so as to average the respective outputs of such humidity sensors adjacent such at least two such mattress coverlet air outlets. For some embodiments, it may be preferred that t least one of such humidity sensors is positioned so as to be situated relatively adjacent an area for support of a patient's hips. In other embodiments, it may be preferred that at least one of such humidity sensors may also include a temperature sensor, for sensing air temperature.

In other of the foregoing embodiments, such coverlet may comprise a low air loss structure, and such apparatus may further include a main patient support structure comprising an air flotation air mattress including its own respective air pump and associated regulator/valving structure. IN some embodiments, such mattress coverlet may be associated with a multi-layer air mattress. In others, such coverlet may comprise a low air loss mattress coverlet having an upper support surface defining a plurality of such air outlets.

In another present exemplary embodiment of the present subject matter, a system may be provided for regulating humidity of air circulating relative to a patient, comprising a low air loss mattress coverlet for association with a main patient support structure, such coverlet defining at least one air inlet thereof and an upper support surface having a plurality of air outlets thereof; a plurality of humidity sensors, coupled respectively adjacent to such mattress coverlet air inlet and such coverlet upper support surface, so as to detect humidity adjacent such air inlet and such upper support surface, and so as to provide respective output signals indicative of each; a coverlet air pump, coupled to such coverlet and configured to move air inside such coverlet from such air inlet thereof through such air outlets thereof; and a controller, receiving such respective output signals of such humidity sensors, and coupled to such coverlet air pump for controlling the humidity of the air moved inside such coverlet by controlling operation of such coverlet air pump.

Yet another present exemplary embodiment relates to a system of closed loop control for regulating humidity of air circulating relative to a patient support. Such system preferably comprises an air flotation air mattress with a plurality of air bladders; an air flotation mattress air pump; air flotation mattress regulator and valving means, for receiving air flow from such air flotation mattress air pump and for regulating inflation of such air bladders of such air flotation air mattress; a low air loss mattress coverlet received on such air flotation air mattress, such coverlet defining at least one air inlet thereof and an upper support surface having a plurality of air outlets thereof; a plurality of humidity sensors, coupled respectively adjacent to such mattress coverlet air inlet and such coverlet upper support surface, so as to detect humidity adjacent such air inlet and such upper support surface, and so as to provide respective output signals indicative of each; a coverlet air pump, coupled to such coverlet and configured to move air inside such coverlet from such air inlet thereof through such air outlets thereof; and a controller, receiving such respective output signals of such humidity sensors, and coupled to such coverlet air pump, for controlling the humidity of the air moved inside such coverlet by controlling operation of such coverlet air pump.

Still further, it is to be understood that present exemplary embodiments equally relate to corresponding methodologies. For example, one present exemplary method relates to methodology for regulating humidity of air circulating relative to a patient support, comprising providing a mattress coverlet for association with a main patient support structure, such coverlet defining at least one air inlet thereof and at least one air outlet thereof; providing a plurality of humidity sensors, coupled respectively adjacent to such mattress coverlet air inlet and such air outlet, so as to detect humidity adjacent such air inlet and air outlet, and so as to provide respective output signals indicative of each; providing a coverlet air pump, coupled to such coverlet and configured to move air inside such coverlet from such air inlet thereof to such air outlet; and providing a controller coupled to such coverlet air pump and such respective output signals of the humidity sensors, for controlling the humidity of the air moved inside such coverlet.

Additional objects and advantages of the present subject matter are set forth in, or will be apparent to those of ordinary skill in the art from, the detailed description herein. Also, it should be further appreciated that modifications and variations to the specifically illustrated, referenced, and/or discussed features, steps, and elements hereof may be practiced in various embodiments and uses of the present subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the present subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the present subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification.

### BRIEF DESCRIPTION OF THE DRAWINGS

A full and enabling disclosure of the present subject matter, including the best mode thereof, directed to one of ordinary skill in the art, is set forth in the specification, which makes reference to the appended figures, in which:

FIG. 1 is a bottom elevational view of an exemplary air flotation mattress in accordance with the present subject matter with exemplary foam bolsters, sides, header, and footer, and individual air cell features of such exemplary mattress running head-to-foot;

FIG. 2 is a cross-sectional view of the exemplary air flotation mattress such as shown in FIG. 1, taken along section line D—D in FIG. 1, showing an exemplary multi-layer mattress coverlet and a multi-layer mattress topper including humidity sensors in accordance with the present subject matter;

FIG. 3 is a top elevational view of the construction of an exemplary mattress coverlet showing exemplary humidity sensor locations in accordance with the present subject matter to aid in moisture regulation during low air loss operation; and

FIG. 4 is a schematic view of exemplary air flotation mattress air cell zones and an external control system which controls their inflation/deflation, and which in accordance with the present subject matter separately provides for independent operation of the subject humidity detection and/or control features.

Repeat use of reference characters throughout the present specification and appended drawings is intended to represent same or analogous features, elements, or steps of the present subject matter.

### DETAILED DESCRIPTION OF THE PREFERRED EMBODIMENTS

As discussed in the Summary of the Invention section, the present subject matter is particularly concerned with apparatus and methodology for controlling the level of moisture within a therapeutic mattresses or mattress coverlet provided in accordance with present subject matter.

Selected combinations of aspects of the disclosed technology correspond to a plurality of different embodiments of the present subject matter. It should be noted that each of the exemplary embodiments presented and discussed herein should not insinuate limitations of the present subject matter. Features or steps illustrated or described as part of one embodiment may be used in combination with aspects of one or more other present embodiment to yield yet further embodiments. Additionally, certain features or steps may be interchanged with similar devices, features or steps not expressly mentioned but which perform the same or similar function.

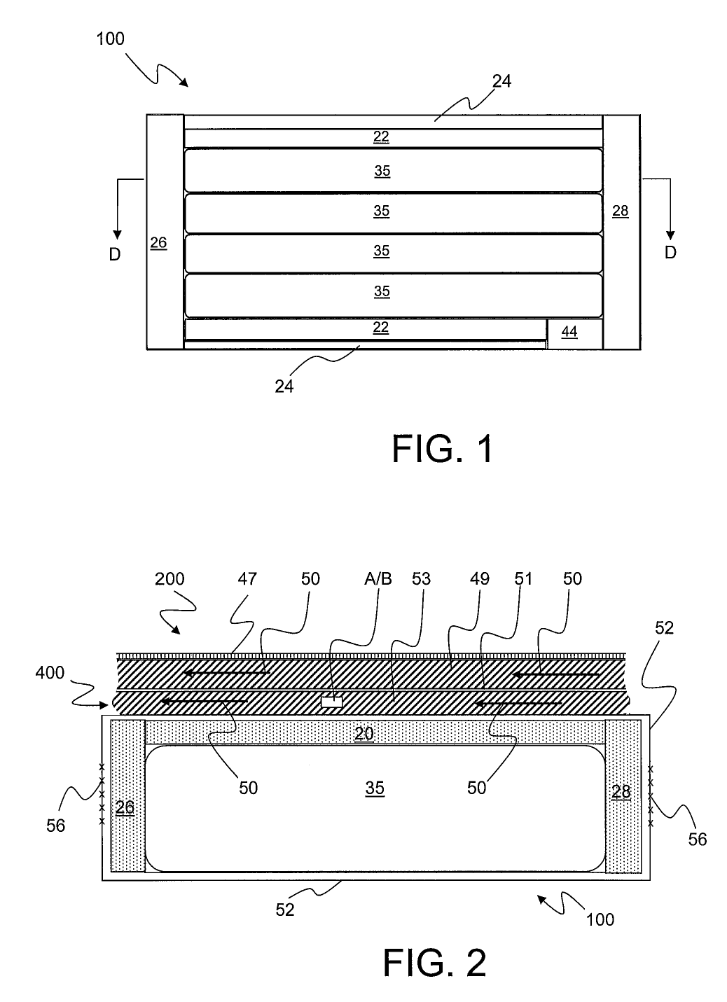

Reference will now be made in detail to exemplary presently preferred embodiments of the subject low air loss moisture removal and control mattress overlay. Referring now to the drawings, FIG. 1 illustrates an exemplary air flotation mattress 100 having a foam shell topper including foam bolsters 22 and foam sides 24 running the length of the mattress 100 and on either side thereof. At the respective ends of the air flotation mattress 100 and capping the foam bolsters and sides 22 and 24 are, respectively, a foam header 26 and foam footer 28, which along with the bolsters 22 form a cavity in the mattress 100. Such cavity (not numbered) is configured for positioning of air cells 35 therein. As may be seen from FIG. 1, a selected plurality of air cells 35 may run from head to foot, received within such cavity.

The cavity formed by the foam bolsters 22, header 26, and footer 28, in accordance with present subject matter, may preferably contain air cells 35, as illustrated. The exemplary air cells 35 are essentially in this instance inflatable air bladders, which are for their alternate inflation/deflation, connected directly to an external control system, as will be more fully described below. Such air cells 35 are operated in a manner so as to provide the primary support surface for a patient.

In accordance with a further exemplary embodiment of the present subject matter, the core of mattress 100 (see, FIG. 2) may be sheathed in/with a multi-layer mattress topper generally 400. The first layer 51 of such multi-layer mattress topper 400 may be a waterproof, vapor impermeable sheet. The second (i.e., middle) layer 53 may comprise a generally non-crush, three-dimensional fabric, such as a knit, cloth, polymeric film, foam or extruded woven fibers. Finally, the third layer 56 may additionally comprise a waterproof, vapor impermeable sheet for protection of the underlying mattress 200. Such third layer 56 may additionally comprise a zippered sheath for encasing the mattress 200. In accordance with the present technology, humidity sensors A, B may be inserted into the second or middle layer 53 as part of the control feedback regarding the humidity level of air flowing through such layer 53, as will be more fully described below.

An exemplary mattress coverlet generally 200 preferably corresponds to its own multi-layer configuration. As seen in FIG. 2, the first layer 46 of such mattress coverlet 200 may be a sheet of waterproof, vapor permeable material. Layer 46 is designed to allow moisture-vapor and heat from a patient's body, or relatively immediately adjacent thereto, to pass through to a second layer 48. Such second layer 48 of mattress coverlet generally 200 corresponds to a non-crush, three-dimensional fabric that is moisture resistant but both vapor and air permeable. It is through such layer 48 of the mattress coverlet 200 that the low air loss features of the present subject matter forces air, which aids in removing the warm moist air generated by the patient. An exemplary depiction of the direction of airflow through the mattress coverlet 200 is indicated by respective exemplary airflow arrows 50 as illustrated in both of layers 48 and 53.

Such two layers 46 and 48 of the mattress coverlet 200 are preferably attached around their perimeters, such as being sewn together. Various methods may be utilized thereafter for further attaching such a coverlet 200. For example, such exemplary coverlet 200 may be formed with an elastic band sewn around its outer perimeter so as to envelop such a mattress 100, as would a fitted sheet.

In the case of a “fitted-sheet” style coverlet 200, the entirety of the outer perimeter of the first and second layers 46 and 48, respectively, may be sewn together. In such an embodiment, the forced air from the external control system along with the warmth and moisture from the air in the second layer 48 of the coverlet 200 may escape around the entire perimeter through the loose friction fit of the elastic band of the coverlet 200.

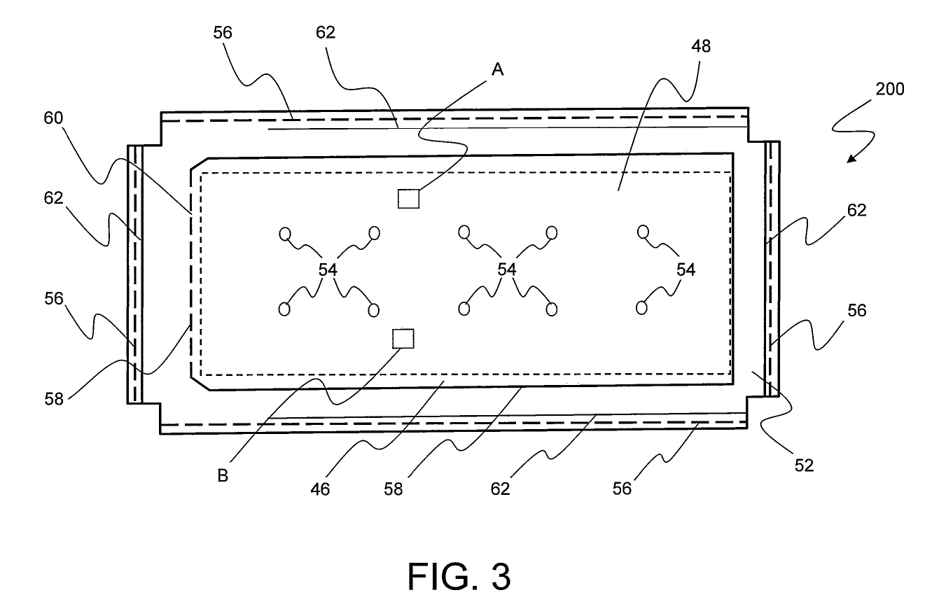

As further represented in the top elevational view of present FIG. 3, billowing may be reduced through the use of spot points of attachment or welds 54 of the first layer 46 of coverlet 200 to the third layer 52 of mattress coverlet 200 in locations throughout the surface of the mattress coverlet 200. In making such spot welds 54, small sections of the material of the second layer 48 of the mattress coverlet 200 have been removed to allow for an unimpeded welding or joinder of the first layer 46 and the third layer 52 of mattress coverlet 200.

As represented, the mattress coverlet 200 first layer 46, in more specific detail, preferably comprises a polyurethane coated polyester which is perimeter welded 58 to the third layer 52 of coverlet 200. Along the designated head end of the coverlet 200, where the first and third layers 46 and 52, respectively, thereof are connected, such perimeter weld 58 is intermittent, so as to resultingly provide for exhaust air ports 60. It is through such exhaust air ports 60 that the warm moist air trapped within the second layer 48 may be disposed.

The third layer 52 of the coverlet 200 preferably comprises a polyurethane coated nylon so as to be moisture and vapor impermeable. The second (i.e., middle) layer 48 is preferably a non-crush three-dimensional fabric. The third layer 52 additionally may have skirt welds 62 along substantially the entire perimeter of the material.

As illustrated in FIG. 3, in an exemplary embodiment of the present technology, a pair of humidity sensors A and B may be placed laterally of a centerline from head-to-foot of the mattress 200 such that the sensors A, B are placed relatively closer to the foot portion of the mattress than the head portion of the mattress, and such that the sensors may be placed in an area proximate an area that may be occupied by the hips of a patient. It should be understood, however, that the sensors A, B may be located anywhere in the airflow path that is not likely to be compressed by a patient to the point of obstruction. Humidity sensors A, B may be connected to an external control system as will be described further below.

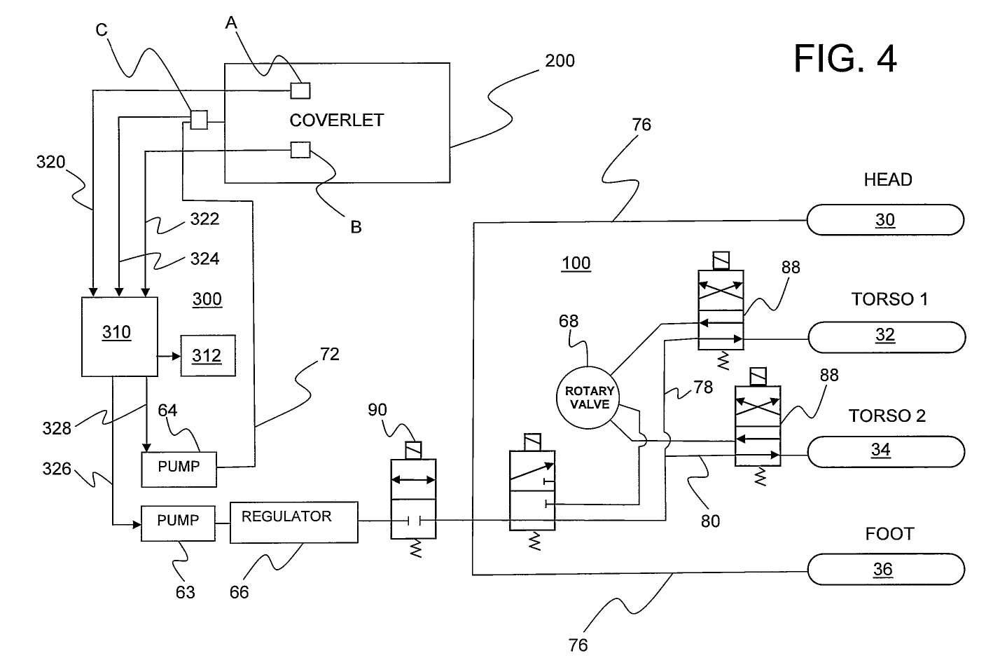

As will be clear to those of ordinary skill in the art from FIG. 4, the air flotation mattress 100 and the mattress coverlet 200 are regulated by an external control system (ECS) 300. The exemplary ECS 300 comprises two pumps 63 and 64, a regulator 66, a rotary valve 68, a single quick-disconnect connector (not illustrated) for connection of air passageway 72 to the mattress coverlet 200, and three quick-disconnect connectors (not illustrated) for respectively connecting air passageways 76, 78, and 80 to the air flotation mattress air cells. Air is provided to both the head and foot zones (30 and 36, respectively) via air passageway 76, and is provided to the two central torso zones 32, 34 via air passageways 78 and 80, respectively. The ECS 300 features are preferably all contained within a stand-alone housing (not illustrated) that may be provided with rubber feet for positioning the housing on the floor and with hooks for hanging the ECS 300 from a bedframe.

In one exemplary embodiment of the present invention including an air flotation mattress with an ECS, the support surface of such air flotation mattress may include in part a foam shell with a surface treatment on its upper surface. An exemplary GEO-MATT.RTM. surface treatment is illustrated in commonly owned U.S. Pat. No. 4,862,538, which is fully incorporated herein by reference, for all purposes. Such surface treatment aids in redistributing skin pressure. Additionally, the air floatation mattress includes a plurality of air cells running side-to-side providing the ability to sub-divide the mattress support into pre-designated zones.

The ECS 300 has two pumps 63 and 64 for separate operation of the air flotation mattress 100 and the mattress coverlet 200. The first pump 63 operates the air flotation mattress 100. It is preferably a pump which provides quiet operation and a quick response to an inflation request. The second pump 64 functions to provide air for the low air loss system in the mattress coverlet 200. The low air loss system pump 64 is preferably a pump which provides a higher air flow rate for the mattress coverlet 200 than would be provided by the air flotation mattress pump 63.

The first pump 63 operates in connection with a regulator 66 and a rotary valve system 68 to provide air for the air flotation mattress 100. In operation of this exemplary embodiment, the air provided to the head and foot zones is delivered through a first passageway 76. This first passageway 76 serves to interconnect the head and foot zones to insure consistent inflation/deflation. The air provided to the torso zone enters through separate passageways 78 and 80, respectively. A pair of control valves 88 are provided with each of the passageways 78 and 80 associated with the torso zones, to either allow inflation/deflation or to maintain the current state of inflation/deflation of the torso zone air cells 32 and/or 34. Such pair of valves 88 are separately operable, which allows for the provision of an alternating pressure support surface within the air flotation mattress 100. When the control valves 88 within passageways 78 and 80 are set to mimic the inflation/deflation of the head and foot zones, the air flotation mattress 100 is able to provide a static support surface. The constructions of such valves 88 and pumps 63 and 64 (as well as the above-referenced quick-disconnect connections) are well known to those of ordinary skill in the art, and details thereof form no particular part of the present subject matter.

The second pump 64 may be operated in accordance with the present subject matter to provide a controllable flow of air to the low air loss mattress coverlet 200. As shown in FIG. 3, the first layer 46 of the mattress coverlet 200 contains air exhaust ports 60 for the expulsion of the low air loss air flow through the mattress coverlet 200. An air input port (not shown) is preferably generally located at the foot end of the mattress coverlet 200 and the air exhaust ports 60 are preferably located at the opposite end of the mattress coverlet 200. However, one of ordinary skill in the art will recognize that alternative configurations of such features fall within the scope and spirit of the present subject matter.

In operation, the ECS 300 functions to provide the user the widest variety of treatment options. The user can select from either a static pressure support surface, in which the air flotation mattress 100 maintains a consistent inflated state across all zones, or an alternating pressure support surface, in which the head and foot zones maintain a consistent inflation state and zones three and four within the torso zone dynamically fluctuate between opposed states of inflation/deflation, respectively. In addition to the choice of support surface function to be provided by the air flotation mattress 100, the ECS 300 allows the user to choose whether or not to allow the operation of the low air loss mattress coverlet 200 to aid in removing warm moist air away from the patient's skin. It is this wide range of user (and/or caregiver) choice in treatment methods and its modularity that allows the system, the air flotation mattress 100, the low air loss mattress coverlet 200 and the ECS 300, to be so flexible.

Additionally, in emergency operations, the system is designed to be as flexible as possible in order to aid in the treatment of the patient. Should the need arise to quickly provide a more sturdy surface for the patient, such as in the case where a patient suffers a heart attack and requires chest compression, the present subject matter provides the user three options: inflate the air flotation mattress 100 fully by utilizing the static support surface feature, terminate the operation of the pumps and allow the air flotation mattress to deflate, or to utilize the quick-disconnect connectors situated between the ECS 300 and the air passageways 76, 78, and 80 to allow for complete deflation of the air flotation mattress 100.

Similarly, when there is a loss of power to the ECS 300, the system is designed to retain its functionality to aid in the treatment of the patient. The air flotation mattress is designed to maintain the inflation pressure within the air cells 30, 32, 34, and 36. It performs such function by allowing the pressure across all the cells 30, 32, 34, and 36 to even out and become consistent (as when utilizing the static pressure support surface feature). The system is able to maintain the air within the cells through the use of the pair of three-way control valves 88 which are opened to allow communication between the air cells 30, 32, 34, and 36 and through the use of a two-way control valve 90 which is closed to deny an exit path for the air already in the system.

Further with respect to FIG. 4, it will be seen that in accordance with the present technology there is provided a controller 310 having input lines 320, 322, 324 configured to couple humidity sensors A, B, C, respectively, to controller 310. As previously mentioned, humidity sensors A and B may be encased within coverlet 200 in an area preferably proximate the area a patient's hips may occupy and are configured to detect moisture levels proximate a patient. Humidity sensor C measures ambient or incoming humidity. Humidity sensor C may be placed in line 72 coupling pump 64 to coverlet 200. Alternatively, humidity sensor C may be placed just inside coverlet 200. In one preferred embodiment of the present subject matter, the net readings between the average of readings from sensors A and B and that of sensor C may be used as a closed-loop control signal that in turn may be used to correspondingly vary the speed of pump 64. Varying the speed of pump 64 in turn varies the moisture change rate (which in this instance is a removal rate). In some embodiments, intended variation of moisture may involve the introduction (rather than removal) of moisture.

It should be appreciated by those of ordinary skill in the art that alternative methodologies for controlling the humidity within coverlet 200 may be provided including, but not limited to, the use of external humidifier and/or dehumidifier apparatus.

In accordance with the present disclosure, yet another exemplary embodiment of the present subject matter may incorporate features responsive to a detected rate of change in humidity (moisture) that may be sensed by humidity sensors A, B (FIG. 4). In particular, the present subject matter provides for monitoring the rate of change in sensed humidity (moisture) so that any sudden increase in detected moisture may be recognized. Such technology has significant value in allowing timely response to incontinence issues as may arise with certain patients.

With reference to FIG. 4, the present technology has provided an exemplary indicator 312 generally illustrated as coupled to controller 310. Indicator 312 may correspond to a number of different devices including, but not limited to, visual and/or audible alarms, and numeric indicators. Indicator 312 may alternatively also correspond to a two part system, including such as a transmitter coupled to controller 310 and a separate remotely located receiver that may provide selected types of visual and/or aural signals to a patient and/or a patient's caregiver.

Implementation of such exemplary present rate of change sensing of moisture detection may correspond to additional circuitry (hardware) or software programming, and/or admixtures of such technology within controller 310. Generally speaking, moisture conditions sensed by humidity sensors A and/or B per present subject matter may be evaluated on an ongoing basis and, for example, compared to either or both of a predetermined fixed or selectively adjustable rate of increase. A relatively rapid rate of increase may be indicative of incontinence issues or of sudden loss of other bodily fluids including, for example, a sudden hemorrhage. In accordance with the present subject matter, selected system operation in response to such sensed conditions may be implemented either as countermeasures to remove such moisture, such as including an increase in air flow, or as other predetermined responses, such as shutting down the air flow system entirely, and/or issuing an alarm to alert the patient and/or caregivers of the detected situation.

In other embodiments, an exemplary coverlet 200 in accordance with the subject invention may be modularly applied to other supports including mattresses, wheelchair/seating cushions, and/or patient positioners (whether air powered, pre-existing, disclosed herewith, or later developed). Several exemplary such support surfaces can be found in commonly owned U.S. Pat. No. 5,568,660 to Raburn et al; U.S. Pat. No. 5,797,155 to Maier et al.; and U.S. Design Patent No. D355,488 to Hargest et al., the disclosures of which are fully incorporated herein by reference, for all purposes.

Additional exemplary embodiments of the present technology may provide temperature sensors in combination with the currently illustrated humidity sensors A, B, and C. The inclusion of such would provide a capability to detect and control relative humidity and/or temperature within coverlet 200. Further embodiments of the present technology may provide humidity sensors with or without temperature sensors in addition to or alternatively to sensors A, B, and C within mattress 100 along with additional controller circuitry to control humidity within mattress 100 in combination with, or alternatively to, humidity control within coverlet 200. The term “humidity” by itself as referenced in this application has been equated to “moisture” content, which is often thought of also as absolute humidity. The term “relative humidity” as referenced herein is its usual meaning, such as defined by the amount of water vapor in a sample of air compared to the maximum amount of water vapor the air can hold at a specific temperature. It's typically expressed as a percentage, from 0% to 100%.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. Methodology for regulating humidity of air circulating relative to a patient support, comprising:
providing a mattress coverlet for association with a main patient support structure, such coverlet defining at least one air inlet thereof and at least one air outlet thereof;
providing a plurality of humidity sensors, coupled respectively adjacent to such mattress coverlet air inlet and such air outlet, so as to detect humidity adjacent such air inlet and air outlet, and so as to provide respective output signals indicative of each;
providing a coverlet air pump, coupled to such coverlet and configured to move air inside such coverlet from such air inlet thereof to such air outlet; and
providing a controller coupled to such coverlet air pump and such respective output signals of the humidity sensors, for controlling the humidity of the air moved inside such coverlet.

2. Methodology as in claim 1, wherein such controller, coverlet air pump, and humidity sensors are arranged so as to comprise a closed loop control system, with such controller configured for adjusting at least one of operation of said coverlet air pump so as to adjust the flow rate of air moved inside said coverlet, and for adjusting the humidity of air output by said coverlet air pump so as to adjust the humidity of air moved inside said coverlet.

3. Methodology as in claim 2, wherein such controller is configured for determining relative humidity of air based on such humidity sensor output signals, and adjusting the humidity of air output by such coverlet air pump so as to adjust the relative humidity of air moved inside such coverlet.

4. Methodology as in claim 2, wherein such controller is configured for determining the rate of change of the humidity of air based on such humidity sensor output signals, for adjusting the output of such coverlet air pump so as to adjust the humidity of air moved inside such coverlet, and for indicating whenever the rate of change of the humidity exceeds predetermined levels, to indicate possible hemorrhaging or incontinence of a patient.

5. Methodology as in claim 1, further including:
providing such coverlet as a low air loss mattress coverlet having an upper support surface defining a plurality of such air outlets;
providing three humidity sensors, with one of such sensors adjacent such mattress coverlet air inlet, and with two of such sensors adjacent such coverlet upper support surface.

6. Methodology as in claim 5, further including averaging the respective outputs of such two humidity sensors adjacent such coverlet upper support surface.

7. Methodology as in claim 1, further including providing a main patient support structure for support of such coverlet, and comprising an air flotation air mattress including its own respective air pump and associated regulator/valving structure.

8. Methodology as in claim 1, further including situating at least one of such humidity sensors relatively adjacent an area for support of a patient's hips.

9. Methodology as in claim 1, further including sensing air temperature of the air being circulated relative to a patient support.
